(19) United States
(12) Patent Application Publication
(10) Pub. No.: US 2014/0106974 A1
(43) Pub. Date: Apr. 17, 2014

(54) PATHOGEN IDENTIFICATION PROCESS AND TRANSPORT CONTAINER

(71) Applicant: SYNBLEX, LLC, State College, PA (US)

(72) Inventors: Yazhou SUN, State College, PA (US); Yeting ZHANG, State College, PA (US)

(73) Assignee: SYNBLEX, LLC, State College, PA (US)

(21) Appl. No.: 13/651,624

(22) Filed: Oct. 15, 2012

(51) Int. Cl. C12Q 1/68; C12Q 1/70

(52) U.S. Cl. CPC C12Q 1/689; C12Q 1/6806; C12Q 1/70
USPC 506/2; 435/6.1; 435/6.15; 435/5; 435/6.12; 435/307.1

(57) ABSTRACT

A process of pathogen identification and a transport container are disclosed. The process includes positioning a porous material containing a sample into an receptacle of a transport container, facilitating transport of the transport container through a mail or parcel service, removing the porous material, the receptacle of the transport container, collecting the sample from the porous material, analyzing the preserved nucleic acid, or a combination thereof. The porous material degrades the sample and preserves nucleic acid. The analyzing identifies a plurality of sequences within the preserved nucleic acid in a parallel manner.

### FIELD OF THE INVENTION

The present invention is directed to methods of transporting pathogens and containers for transporting samples, such as pathogens. More specifically, the present invention is directed to such methods and containers capable of use with degraded samples having preserved nucleic acid.

### BACKGROUND OF THE INVENTION

Increased globalization continues to result in increased international and intercontinental travel. Increased interpersonal and societal dynamics, along with increased travel, greatly increase pathogen dispersion and transmission. This leads to an increased number of food-borne illness outbreaks and socially/economically debilitating epidemics and pandemics, for example, the introduction and proliferation of HIV/AIDS, the SARS outbreak in Asia in 2003, multiple deadly Escherichia coli, Listeria and Salmonella outbreaks, H1N1 flu pandemic, cholera epidemics in Zimbabwe and Haiti, and the manmade anthrax attacks. As a consequence, bacterial and viral pathogens are of particular concern to public health, medical care, and national security.

To prevent epidemics and pandemics, effective infectious disease surveillance, screening, and treatment are necessary. Although currently there are effective treatments or preventions against many pathogens, there are severe limitations in rapid and convenient pathogen screening methods and technologies: the requirement of a viable pathogen sample, the risks associated with transporting live pathogen, the lack of thoroughness, and the requirement of highly trained professionals.

There are two major categories of detection methods currently available on the market for pathogen detection: protein-based and nucleic acid-based. Protein-based methods utilize antibodies that can recognize and bind to specific antigens from certain pathogens for which these antibodies are designed. This is usually referred to as immunoassay-based method. These antibodies are usually labeled with, for example, a type of fluorescent dye that produces a measurable signal after the binding between antibody and antigen occurs.

Nucleic acid-based methods, mostly DNA-based, usually utilize sequence-specific polymerase chain reaction (PCR) amplification that targets a given pathogen's unique DNA (or RNA) signature with specific primers. This is usually referred to as PCR-based method. The successful amplification could be measured by electrophoresis band at the appropriate size range, or more accurately, by direct sequencing that decodes the DNA nucleotide sequence.

The application of either method requires information to be known about a predetermined pathogen in order to select the correct antibodies or primers. This requires certain expectations as to which pathogens may be present and should be tested by the users before submitting the samples for testing, which makes these tests expectation-independent. For example, physicians might expect certain types of bacteria to be present in a collected sample by knowing certain symptoms of the patients where the sample is collected and by having knowledge that such symptoms are often associated with such types of bacteria. In practice, this means that pathogens must be expected to be present in the sample, either based upon user experiences or expert knowledge, prior to looking for them. Such expectations may not be practical or even possible under realistic circumstances, for example, due to lack of prior knowledge, abnormal symptoms, or other factors. In addition, in order to screen against multiple potential pathogens in one sample, multiple tests are required where each test is specific to one type of pathogen. As a result, the cost is high and the complexity is great. Broad-spectrum screening, which refers to screening against a broad range of different substances, using these methods also requires large amounts of viable sample, which sometimes is impractical or impossible. Both immunoassay-based and PCR-based methods have these disadvantages, so they are only suitable for targeting a few known pathogens that are already suspected by the clients.

As a result, there is an unmet need for products and services that are capable of detecting a wide spectrum of pathogens rapidly and conveniently. Recent development in pathogen detection technologies, especially with nanotechnology and microfludics, has dramatically increased the speed and throughput in pathogen detection while at the same time has decreased the required amount of sample and reagents. For example, microfludics biosensors are capable of detecting even only a few pathogen cells while consuming a fraction of reagents compared with traditional methods. Also, nucleic acid microarrays, which are tiny plates coated with hundreds of thousands of nucleic acid probes that are each designed for a specific region of a known type of pathogen, can screen against many pathogens at the same time.

However, these methods still have severe limitations that hinder their wider adoption. Any biosensors that are protein-based require the pathogens in the sample to be viable or the targeted proteins are not degraded. However, these requirements demand complex procedures and specialized equipment during sampling and transportation. For example, to avoid protein degradation or maintain pathogen viability, wet/dry ice shipping is one of the minimal requirements. Because the pathogens are sampled, transported, and delivered in a viable state, extreme cautions are needed to avoid accidental contamination, both to the sample and to the environment. While biosensors are able to provide straightforward results, screening against a broad-spectrum of pathogens still means stacked-up cost and complexity.

Microarrays are based on a different reaction principle, which is hybridization between probes on the microarray plate and nucleic acid fragments from targeted pathogens in the sample. This is different from amplification. Therefore microarrays require the presence of a higher number of nucleic acid fragments in the sample in order to produce measurable signals. This means a large sample volume, which is normally achieved by culturing in the lab after the sample is received. This again demands that the pathogens are maintained viable throughout transportation.

One solution to reduce the risks associated with transporting viable pathogens is to eliminate the transportation step. To achieve this, a mobile or on-site lab capable of performing the pathogen detection is necessary. However, this not only requires costly and specialized equipment, but also requires professionally trained and highly paid lab technicians to be on-site and to carry out the procedures.

In addition to the expenses and complexities of transporting biological samples while maintaining the viability of the pathogens or other organisms within the samples, and risks of accidental exposure of the pathogens to the environment and accidental contamination to the samples from the environment, transporting samples containing live pathogens or other living organisms have other significant ramifications. Among them are bioterrorism and sabotage acts. Terrorists could transport damaging pathogens to the analyzing lab or other facilities deliberately without any notification or marking Opening the containers with these damaging pathogens under no or inadequate biosafety protection procedures and/or equipment by the unsuspecting lab staff will spread these pathogens to the facility staff and then further to the general public. Such events occurred in the United States during the year 2001 with anthrax. Additionally, since viability is maintained thorough the transportation process, attackers could intentionally add other pathogens or other damaging organisms into the sample during the transportation process and the added pathogens will also be kept alive by the conditions (for example, low temperature) created for maintaining sample viability. Or the attackers could easily remove the conditions that are necessary to maintain sample viability and make the downstream pathogen detection difficult.

Pathogen identification process and transport containers that do not suffer from one or more of the above drawbacks would be desirable in the art.

### BRIEF DESCRIPTION OF THE INVENTION

In an exemplary embodiment, a process of pathogen identification includes positioning a porous material containing a sample into a receptacle of a transport container, and facilitating transport of the transport container through a mail or parcel service. The porous material degrades the sample and preserves nucleic acid.

In another exemplary embodiment, a process of pathogen identification includes removing a porous material containing a sample from a receptacle of a transport container. The sample is degraded and has preserved nucleic acid.

In another exemplary embodiment, a process of pathogen identification includes collecting a sample, the sample being degraded and having preserved nucleic acid, and analyzing the preserved nucleic acid. The analyzing identifies a plurality of sequences within the preserved nucleic acid in a parallel manner.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 shows a pictorial representation of an exemplary pathogen identification process according to the disclosure.

FIG. 2 shows an exemplary transport container prior to the porous material being positioned within it and a front and rear view of the transport container upon the porous material being positioned within it according to the disclosure.

FIG. 3 shows a schematic representation of an exemplary pathogen identification process according to the disclosure.

FIG. 4 is a plot of bioanalyzer results for a comparative example collection of DNA directly from a fermented food biomass source, with amplification of bacterial 16S rRNA V3 (338-514).

FIG. 5 is a plot of bioanalyzer results for an exemplary collection of DNA using FTA cards from a fermented food biomass source having bacterial 16S rRNA V3 (338-514) according to the disclosure.

FIG. 6 is a plot of bioanalyzer results for a comparative example collection of DNA directly from a fermented food biomass source, with amplification of bacterial 16S rRNA V6 (781-1082, also including V5 and V7).

FIG. 7 is a plot of bioanalyzer results for an exemplary collection of DNA using FTA cards from a fermented food biomass source having bacterial 16S rRNA V6 (781-1082, also including V5 and V7) according to the disclosure.

FIG. 8 is a plot of bioanalyzer results for a comparative example collection of DNA directly from a human saliva source, with amplification of bacterial 16S rRNA V3 (338-514).

FIG. 9 is a plot of bioanalyzer results for an exemplary collection of DNA using FTA cards from a human saliva source having bacterial 16S rRNA V3 (338-514) according to the disclosure.

FIG. 10 is a plot of bioanalyzer results for a comparative example collection of DNA directly from a human saliva source, with amplification of bacterial 16S rRNA V6 (781-1082, also including V5 and V7).

FIG. 11 is a plot of bioanalyzer results for an exemplary collection of DNA using FTA cards from a human saliva source having bacterial 16S rRNA V6 (781-1082, also including V5 and V7) according to the disclosure.

Wherever possible, the same reference numbers will be used throughout the drawings to represent the same parts.

### DETAILED DESCRIPTION OF THE INVENTION

Provided is an exemplary pathogen identification process and a transport container. Embodiments of the present disclosure permit rapid screening of a broad range of pathogens, permit screening of pathogens without previously specifying the pathogen to test for, permits screening of all pathogens with known nucleotide sequences at once, permits unskilled and/or uneducated individuals to collect samples, decreases risk of harm from samples (for example, by rendering samples non-viable), decreases risk of contamination of samples, permits samples to be analyzed at lower costs than on-site analysis or analysis requiring a viable sample, increases the chances of responding adequately to outbreaks, or combinations thereof.

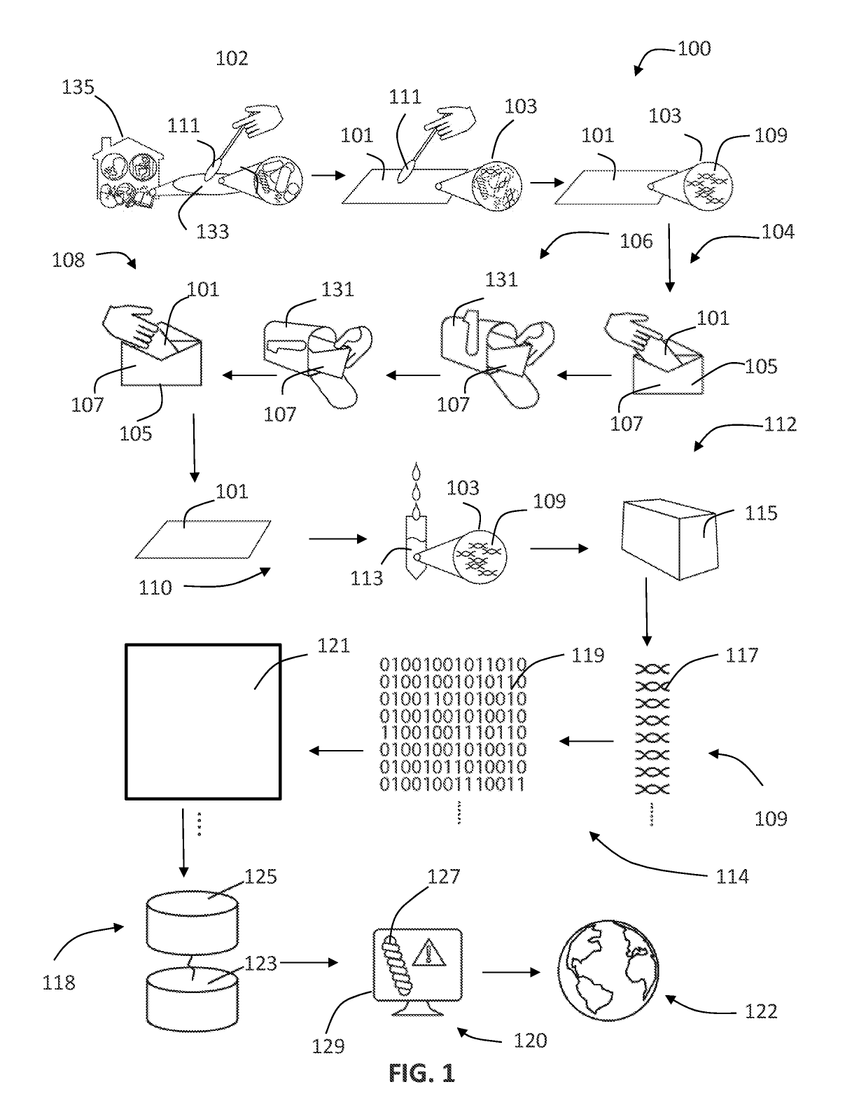

Referring to FIG. 1, a process 100 of pathogen identification includes any suitable combination of steps for safe and effective handling of pathogens. For example, in one embodiment, the process 100 includes positioning a sample 103 onto and/or within a porous material 101 (step 102), placing the porous material 101 into a receptacle 105 of a transport container 107 (step 104), facilitating transport of the transport container 107 through a mail or parcel service 131 (step 106), removing the porous material 101 from the receptacle 105 of the transport container 107 (step 108), collecting the sample 103 from the porous material 101 (step 110), amplifying nucleic acid 109 from the sample 103 (step 112), analyzing nucleic acid 109 from the sample 103 (step 114), or a combination thereof. As will be appreciated by those skilled in the art, one or more of the steps of the process 100 are capable of being included or excluded, depending upon identified standard operating procedures.

The porous material 101 is any suitable material capable of receiving the sample 103. In one embodiment, the porous material 101 is selected from the group consisting of cellulosic material (for example, paper, cardboard, or filter paper), fibers (for example, microfibers or nanofibers), fiberglass, cloth, and combinations thereof. In one embodiment, the porous material 101 is an FTA card (available from GE Healthcare, Waukesha, Wis.). For example, in one embodiment, the porous material 101 is a treated fiber matrix having chemicals to lyse, to degrade proteins, to preserve the nucleic acid 109, or a combination thereof. In one embodiment, such lysing includes lysing cell membranes, lysing cell walls, lysing of protein, lysing of a cellular or organelle membrane, lysing of lipids, degrading of a viral capsule, and combinations thereof. The sample 103 is any material having the nucleic acid 109, for example, cells, pathogens, non-pathogens, viruses, bacteria, microorganism, human cells, non-human cells, or combinations thereof. In one embodiment, the sample 103 is derived from a pathogen capable of airborne contamination. In one embodiment, the sample 103 is derived from a bodily substance selected from the group consisting of saliva, sputum, urine, blood, blood stains, skin, fecal matter, and combinations thereof, and/or another substance, such as, water, food, plants, industrial materials, soil, waste, surface materials, food, drink, fermented biomass, environmental water, ecological materials, viruses, bacteria, or combinations thereof. The nucleic acid 109 includes deoxyribonucleic acid (DNA), ribonucleic acid (RNA), transfer RNA (tRNA), messenger RNA (mRNA), ribosomal RNA (rRNA), non-coding RNA (ncRNA), non-protein-coding RNA (npcRNA), non-messenger RNA (nmRNA), functional RNA (fRNA), small RNA (sRNA), small nucleolar RNA (snoRNA), short RNA (microRNA or miRNA), small interfering/short interfering/silencing RNA (siRNA), piwi interacting RNA (piRNA), long non-coding RNA (long ncRNA), or a combination thereof.

The positioning of the sample 103 onto and/or within the porous material 101 (step 102) is capable of being performed on-site, for example, from a surface 133, at an event location 135 (for example, a crash site, a location of outbreak, a quarantine zone, a remote hospital, a remote village, or a combination thereof), within a mobile lab (for example, a lab having little or no analytical instrumentation), in high-traffic locations (for example, an airport, a train station, a bus station, a university, an office, a city, a government building, a tourist destination, or a combination thereof), any other suitable location, or a combination thereof. In one embodiment, the positioning (step 102) includes fixating the sample 103 onto the porous material 101, for example, based upon a set of instructions and/or by an individual with little or no training and/or education. In one embodiment, the positioning (step 102) is performed by using a swab 111 and/or by applying one or more drops of the sample 103 onto the porous material 101. Upon positioning the sample 103 onto and/or within the porous material 101 (step 102), the sample 103 is degraded, killed, or otherwise rendered non-viable, while the nucleic acid 109 of the sample 103 is preserved. The sample 103 is degraded, killed, or otherwise rendered non-viable by the porous material 101 and/or by on-site techniques capable that preserve the nucleic acid 109, for example, by using ethanol or isopropanol.

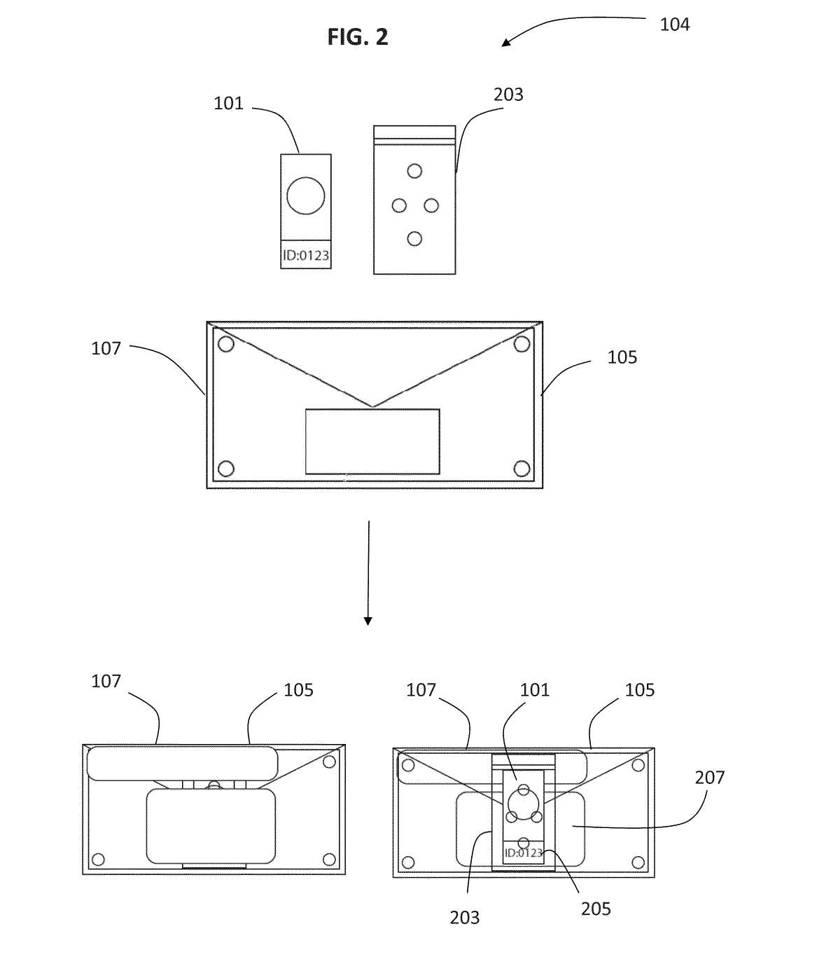

The placing of the porous material 101 into the receptacle 105, such as an envelope, sleeve, and/or folder, of the transport container 107 (step 104) is dependent upon the transport container 107 utilized. Referring to FIG. 2, the transport container 107 includes the receptacle 105 capable of containing the porous material 101. In some embodiments, the transport container 107 includes a sample enclosure 203, such as a smaller envelope, sleeve, and/or folder.

The transport container 107, the receptacle 105, and/or the sample enclosure 203 include(s) any suitable features for security, identification, and/or safety. Suitable features include, but are not limited to, allowing easy visual inspection of labeling 205, allowing easy visual inspection of the sample 103 on the treated porous material 101 within the transport container 107, allowing easy visual inspection of identification information of the sample 103 within the container 107, allowing easy visual inspection of other foreign or suspicious matters within the container 107, or a combination thereof, while the receptacle 105, the transport container 107, the receptacle 105, and/or the sample enclosure 203 is/are closed. In a further embodiment, such security, identification, and/or safety is, at least in part provided by having a signification transparent or clear portion in the container 107, being devoid or substantially devoid of internal compartments within the container 107, having internal organization of the container 107 to make all content clearly and easily visible from outside, other suitable features, or combinations thereof. Additionally or alternatively, in one embodiment, a security seal 207 capable of indicating whether the transport container 107, the receptacle 105, and/or the sample enclosure 203 has/have been opened is in the transport container 107.

In one embodiment, the transport container 107, the receptacle 105, and/or the sample enclosure 203 permit(s) moisture and air to flow into and out of the transport container 107, the receptacle 105, and/or the sample enclosure 203 and/or is not completely sealed. Additionally or alternatively, the transport container 107, the receptacle 105, and/or the sample enclosure 203 include(s) a desiccant (not shown) and/or exclude(s) wet or dry ice. This is to reduce the viability of pathogens or other damaging biological agents introduced by potential bioterrorist or saboteur.

In one embodiment, the transport container 107 prevents direct physical contact with the porous material 101 and/or prevents direct physical contact with the sample 103, for example, by having an inner transparent envelope with a first set of one or more openings (not shown) and an outer transparent envelope with a second set of one or more openings (not shown), wherein the first set and the second set are arranged in a non-overlapping manner, regardless of the positioning of the inner envelope inside the outer envelope.

Referring again to FIG. 1, in placing the porous material 101 into the receptacle 105 of the transport container 107 (step 104), any suitable procedures for preserving the sample 103 and maintaining safety are employed. For example, in one embodiment, the porous material 101 is only positioned in non-watertight and non-airtight conditions, is positioned with information about the sample 103 being legible without opening the transport container 107, is positioned in a manner permitting the sample 103 to be viewed without opening the transport container 107, or a combination thereof.

The facilitating of transport of the transport container 107 through the mail or parcel service 131 (step 106) includes directing such transport, performing such transport, overseeing such transport, or a combination thereof. In one embodiment, the transport is between sites (for example, over a distance of greater than one mile), between cities (for example, over a distance of greater than twenty miles), between countries (for example, over a distance greater than 100 miles, 500 miles, 1,000 miles, or any range therein), overseas (for example, over a distance greater than 3,000 miles), or any other suitable distance within a predetermined period (for example, within one hour, one day, three days, one week, one month, or any range therein).

The removing of the porous material 101 from the receptacle 105 of the transport container 107 (step 108) occurs upon the transport container 107 arriving at a predetermined location, such as a lab facility (not shown). The removing (step 108) occurs under any suitable operating procedures, for example, in a designated and secured area, with priority designations based upon information capable of being viewed without opening the transport container 107, based upon timing of the arrival of the transport container 107, with any suitable automated mail sorting techniques, or a combination thereof.

The collecting of the sample 103 from the porous material 101 (step 110) includes extracting the nucleic acid 109 from the porous material 101 into a sample solution 113. Any suitable extraction technique may be used, for example, FTA purification reagents and accessories (available from GE Healthcare, Waukesha, Wis.), QIAamp DNA Mini Kit and accessories (available from QIAgen, Venlo, Netherlands), Agencourt beads, kits and accessories (available from Beckman Coulter, Danvers, Mass.), Omega Bio-Tek Genomic DNA isolation kits and accessories (available from Omega Bio-Tek, Norcross, Ga.), or a combination thereof.

The amplifying of the nucleic acid 109 from the sample 103 (step 112), if performed, increases the amount of the nucleic acid 109 without culturing of the sample 103. The amount of the nucleic acid 109 is about 100 nanograms, 20 nanograms, 1 nanogram, or any range therein. Increasing the amount of the nucleic acid 109 permits use of a wider range of techniques for the analyzing (step 114) of the sample 103. In one embodiment, the amplifying (step 112) is targeted and includes using custom primers (not shown), for example, primers used for 16s rRNA, primers targeting known antibiotic-resistance genes, primers used for pathogenicity island genes, amplicon primers capable of targeting a wide range of pathogens, other suitable primers, or a combination thereof (an amplicon is an amplified DNA from a specific amplification process, for example, from PCR using a pair of primers). Design and selection of such primers are performed under any suitable conditions, for example, selecting conserved primers between highly-variable regions to provide species or strain-level resolution and/or selecting conserved regions representing the same pathogenic or antibiotic-resistant markers between different species, both of which rely upon a thorough sequence conservation survey for regions of interest. Suitable sequence conservations survey methods include, but are not limited to, alignment by using Smith-Waterman algorithm, using sequence similarity search tools, such as a Basic Local Alignment Search Tool or BLAST (available from the National Center for Biotechnology Information, Bethesda, Md.), using k-mer indexing (for example, as is used with next generation sequencing short read mapping), or a combination thereof. The use of such primers is performed under predetermined conditions, such as within a predetermined temperature range, with a predetermined buffer composition and a predetermined ion concentration, based upon the primer design process, the chemical properties of the primer oligonucleotide, the amplification reaction system, or a combination thereof.

The analyzing of the nucleic acid 109 from the sample 103 (step 114) includes use of a sequencer 115, for example, capable of multiplex PCR and/or high-throughput parallel sequencing, permitting the nucleic acid 109 of different sources (not shown) in the sample 103 to be distinguished. Additionally or alternatively, the sequencer 115 individually sequences for the nucleic acid 109 of the different sources (not shown) in the sample 103. Due to the absence of culturing, the analyzing (step 114) is capable of being completed in a shorter duration of time, such as, within 8 hours, within 2 hours, within 1 hour, or any suitable range therein.

In one embodiment, the sequencer 115 is a high-throughput DNA sequencer, also known as a Next Generation Sequencer (for example, MiSeq from Illumina, Inc. of San Diego, Calif. or Ion Torrent from Life Technologies, of Guilford Conn.), capable of generating millions of sequencing reads by simultaneously sequencing each nucleic acid molecule in the sample 103 in parallel manner, for example, permitting testing of all known sequences in a single test.

In one embodiment, the analyzing of the nucleic acid 109 from the sample 103 (step 114) includes decoding each molecule 117 of the nucleic acid 109 (step 116) into digital character strings 119 that correspond with nucleotide sequences 121. The nucleotide sequences 121 are capable of being stored as the digital character strings 119 (each of which may also be known as a read 123). In one embodiment, the sequencer 115 generates more than one million of the reads 123 for the sample 103.

In one embodiment, the reads 123 are compared with a reference database 125 (step 118), for example, containing information about all currently known sequences, corresponding species, pathogenicity, other useful information, or a combination thereof. If a match between the reference database 125 and one of the reads 123 occurs, then the read 123 is categorized as being from a corresponding known source (not shown). If there are N number of the reads 123 that are determined as being from the corresponding known source, the relative concentration of a source in the sample 103 is capable of being determined as NIT, where T is the total number of the reads 123 from the sample 103, for example, in an automated manner and/or with frequent updates to the reference database 125 (such as, daily, weekly, monthly, quarterly, or yearly). In one embodiment, the sample 103 is re-analyzed upon updating of the reference database 125 by reanalyzing digital information based upon the reads 123 from the sequencer 121, for example, up to any suitable period where the information corresponding to the sample 103 remains valuable, independent of whether the sample 103 remains present or intact (for example, one week, one month, three months, one year, or any other suitable period).

If a match between the reference database 125 and the read 123 does not occur, the information that such an unknown sequence is present in the sample 103 is capable of being used for identification and/or investigation purposes. For example, the same unknown sequence from different sources, such as patients sharing similar symptoms, may suggest the presence of a new microbiome species, such as an infectious agent responsible for a disease. In addition, such unknown sequence information is capable of being used to provide correlation and/or relationship information between different samples sharing similar characteristics and/or future identification, for example, upon subsequent updating of the reference database 125.

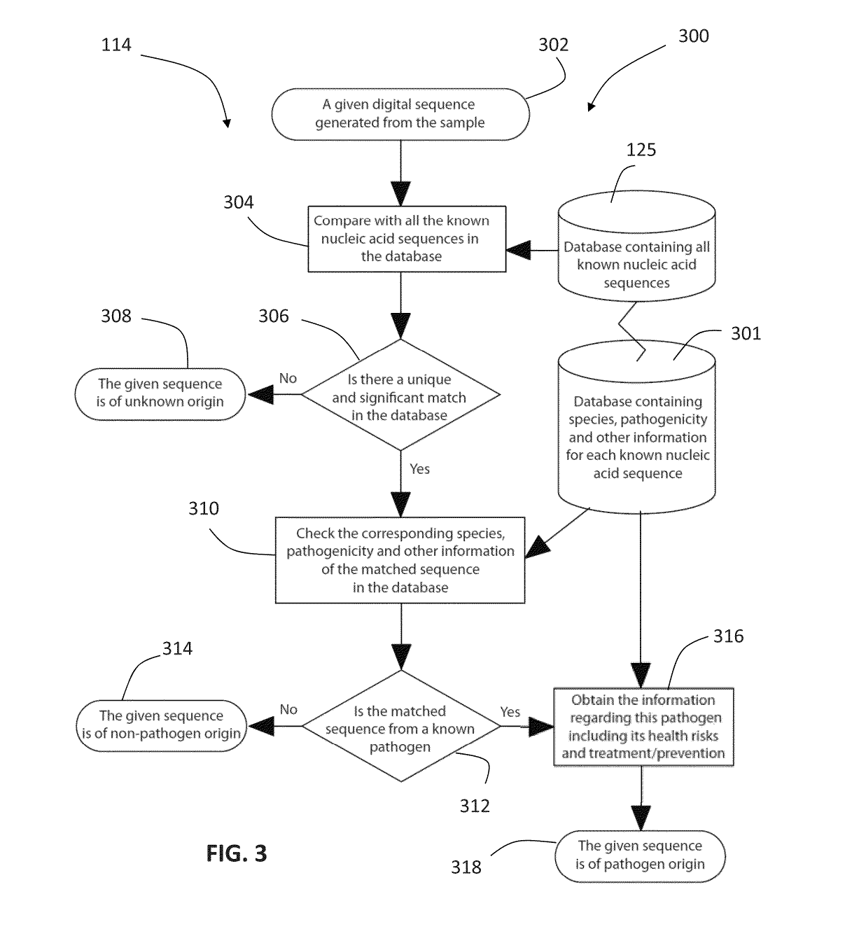

Referring to FIG. 3, in one embodiment, the analyzing (step 114) is based upon a predetermined algorithm 300 for identifying whether the sequence 121 is of pathogen origin. For example, in this embodiment, the algorithm 300 begins by generating a digital sequence (step 302), then comparing the digital sequence with known nucleic acid sequences in the database 125 (step 304). A determination of whether there is a unique and significant match is then made (step 306). If no match is made, then the digital sequence is identified as having an unknown origin (step 308). If a match is made, then the digital sequence is correlated to information 301 about the digital sequence (step 310), for example, information about a species, pathogenicity, other helpful information, or a combination thereof. A determination as to whether the digital sequence, is of a pathogen source, is then made (step 312). If the information corresponds with a known source that is not a pathogen, then the digital sequence is identified as being of non-pathogen origin (step 314). If the information corresponds with a source that is known as a pathogen, then information is obtained regarding the pathogen (step 316) and the digital sequence is identified as being from the pathogen (step 318).

Referring again to FIG. 1, in one embodiment, results 127 of the analyzing (step 114) are displayed in any suitable manner (step 120). In one embodiment, the results 127 are displayed (step 120) on a device 129, such as, a computer, a mobile device, a phone, any device having communication access (for example, having internet access, cell tower access, and/or satellite access), or a combination thereof. In a further embodiment, the device 129 permits the information to be transmitted to and received in any accessible location (step 122), for example, upon secure access to a web portal. The results 127 are capable of being displayed in any suitable medium, include descriptions of the sample 103, include health risks of the sample 103, include procedures for handling the sample 103, include concentrations of the sample 103, include any other useful information, or a combination thereof.

### EXAMPLES

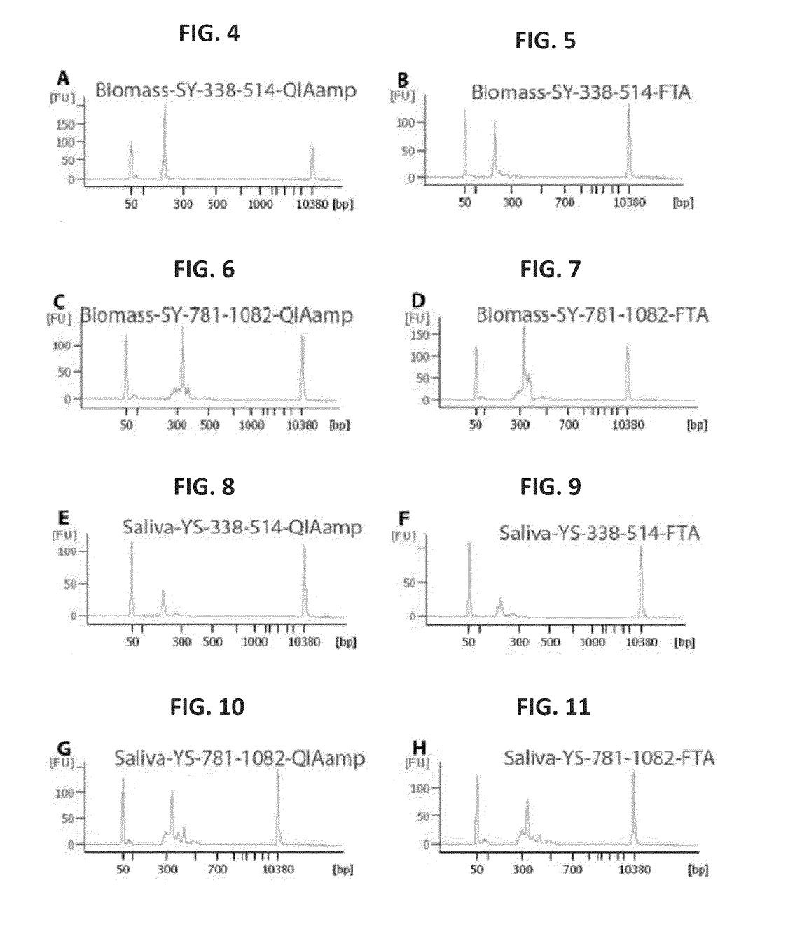

A first example, a comparative example, includes collection of DNA directly from a fermented food biomass source, with amplification of bacterial 16S rRNA V3 (338-514). Analysis of the first example shows three peaks as are shown in FIG. 4.

A second example includes collection of DNA using FTA cards from the fermented food biomass source in the first example, according to an embodiment of the disclosure, having the bacterial 16S rRNA V3 (338-514). Analysis of the second example shows three peaks as are shown in FIG. 5. The peaks are consistent with the peaks of the first example, with slightly different intensity.

A third example, a comparative example, includes collection of DNA directly from a fermented food biomass source, with amplification of bacterial 16S rRNA V6 (781-1082, also including V5 and V7). Analysis of the third example shows three peaks as are shown in FIG. 6.

A fourth example includes collection of DNA using FTA cards from the fermented food biomass source in the third example, according to an embodiment of the disclosure, having the bacterial 16S rRNA V6 (781-1082, also including V5 and V7). Analysis of the fourth example shows three peaks as are shown in FIG. 7. The peaks are consistent with the peaks of the third example, with slightly different intensity.

A fifth example, a comparative example, includes collection of DNA directly from a human saliva source, with amplification of bacterial 16S rRNA V3 (338-514). Analysis of the fifth example shows three peaks as are shown in FIG. 8.

A sixth example includes collection of DNA using FTA cards from the human saliva source in the fifth example, according to an embodiment of the disclosure, having the bacterial 16S rRNA V3 (338-514). Analysis of the sixth example shows three peaks as are shown in FIG. 9. The peaks are consistent with the peaks of the fifth example, with slightly different intensity.

A seventh example, a comparative example, includes collection of DNA directly from a human saliva source, with amplification of bacterial 16S rRNA V6 (781-1082, also including V5 and V7). Analysis of the seventh example shows three peaks as are shown in FIG. 10.

A eighth example includes collection of DNA using FTA cards from the human saliva source in the seventh example, according to an embodiment of the disclosure, having the bacterial 16S rRNA V6 (781-1082, also including V5 and V7). Analysis of the eighth example shows three peaks as are shown in FIG. 11. The peaks are consistent with the peaks of the seventh example, with slightly different intensity.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. A process of pathogen identification, the process comprising:
collecting a sample with a porous material;
positioning the porous material containing the sample into a receptacle of a transport container, the porous material degrading the sample and preserving nucleic acid;
facilitating transport of the transport container to a predetermined location through a mail or parcel service;
analyzing the preserved nucleic acid at the predetermined location using high-throughput sequencing; and
transmitting results of the analysis to a device having communication access;
wherein the porous material degrades the sample and preserves nucleic acid;
wherein facilitating transport is selected from the group consisting of directing such transport, performing such transport, overseeing such transport, and combinations thereof; and
wherein the high-throughput sequencing simultaneously sequences each preserved nucleic acid molecule in the sample;
wherein the process permits the detection of any known pathogens in the sample in one test without the requirement of expecting which pathogens are present in the sample prior to the testing.

23. (canceled)

24. (canceled)

25. (canceled)


SEQUENCE LISTING (7 sequences)

SEQ ID NO: 1 - DNA, 1542 bp, Escherichia coli
  DNA (1)..(1542): Strain K12 Sub-strain DH10B
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcttttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 2 - RNA, 156 nt, Escherichia coli
  rRNA (1)..(156): V3 region of 16S rRNA
ccccuuauaacguguuacccgcguucggacuacgucgguacggcgcacauacuucuuccggaagcccaacauuucaugaaagucgccccuccuucccucauuucaauuauggaaacgaguaacugcaaugggcgucuucuucguggccgauugagg

SEQ ID NO: 3 - RNA, 274 nt, Escherichia coli
  rRNA (1)..(274): V6 (including V5 and V7) region of 16S rRNA
gcggcauuugcuacagcugaaccuccaacacgggaacuccgcaccgaaggccucgauugcgcaauucagcuggcggaccccucaugccggcguuccaauuuugaguuuacuuaacugcccccgggcguguucgccaccucguacaccaaauuaagcuacguugcgcuucuuggaauggaccagaacuguaggugucuugaaaggucucuaccuaaccacggaagcccuugacacucuguccacgacguaccgacagcagucgagcacaacacuu

SEQ ID NO: 4 - DNA, 21 bp, Escherichia coli
  primer_bind (1)..(21): Primer binding site for V3 region of 16S rRNA
actcctacgggaggcagcagt

SEQ ID NO: 5 - DNA, 21 bp, Escherichia coli
  primer_bind (1)..(21): Primer binding site for V3 region of 16S rRNA
gtattaccgcggctgctggca

SEQ ID NO: 6 - DNA, 27 bp, Escherichia coli
  primer_bind (1)..(27): Primer binding site for V6 region of 16S rRNA
aacaggattagataccctggtagtcca

SEQ ID NO: 7 - DNA, 24 bp, Escherichia coli
  primer_bind (1)..(24): Primer binding site for V6 region of 16S rRNA
ctcgttcgggacttaacccaacat